(19) United States
(12) Patent Application Publication
(10) Pub. No.: US 2009/0062536 A1
(43) Pub. Date: Mar. 5, 2009

(54) USE OF SULFONANILIDE COMPOUNDS AS HERBICIDE

(75) Inventors: KOICHI ARAKI, Ushiku-shi (JP); Yoshitaka Sato, Tsukuba-city (JP); Shinichi Shirakura, Oyama-shi (JP); Keiji Endo, Inashiki-gun (JP); Shin Nakamura, Oyama-shi (JP); Seiji Ukawa, Oyama-shi (JP); Chieko Ueno, Oyama-shi (JP)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Bayer CropScience AG

(21) Appl. No.: 12/193,236

(22) Filed: Aug. 18, 2008

(30) Foreign Application Priority Data
Aug. 20, 2007 (JP) 2007-213330

(51) Int. Cl. C07D 251/30; C07D 239/48

(52) U.S. Cl. 544/219; 544/319

(57) ABSTRACT

A herbicide comprising as an active ingredient a sulfonanilide compound represented by the formula:

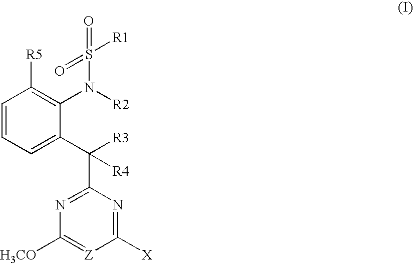

wherein,
R1 represents CHF₂ or CH₂CF₃; R2 represents hydrogen, C1-3 alkyl, 3-propenyl, or 3-propynyl; R3 represents hydrogen; R4 represents hydrogen, hydroxy, or methylthio, or R3 and R4, together with a carbon atom to which they are bonded, may form C═O; R5 represents halogen or methyl; X represents methoxy or chlorine; and Z represents CH or N;
   with the proviso that,
(i) when R1 represents CH₂CF₃, R2 represents hydrogen, R5 represents bromine or iodine, X represents methoxy, and Z represents CH,
(ii) when R1 represents CHF₂ and X represents methoxy, R5 represents bromine or iodine, Z represents N, and R2 represents C1-3 alkyl, 3-propenyl, or 3-propynyl,
(iii) when R1 represents CHF₂ and X represents chlorine, Z represents CH.

### TECHNICAL FIELD

The present invention relates to use of sulfonanilide compounds as herbicides, new sulfonanilide compounds, and a preparation method and intermediates thereof.

### BACKGROUND ART

Some kinds of sulfonanilide compounds have been known to have an activity as herbicides (see, e.g., Patent Documents 1-6), and sulfonanilide compounds also have been known to have an activity as bactericides (e.g., Patent Document 7)

[Patent Document 1] a pamphlet of PCT WO 93/09099

[Patent Document 2] a pamphlet of PCT WO 96/41799

[Patent Document 3] Japanese Patent Application Laid-open No. 11-60562

[Patent Document 4] Japanese Patent Application Laid-open No. 2000-44546

[Patent Document 5] Japanese Patent Application Laid-open No. 2006-56870

[Patent Document 6] Japanese Patent Application Laid-open No. 2007-106745

[Patent Document 7] Japanese Patent Application Laid-open No. 2006-56871

### DISCLOSURE OF INVENTION

In developing herbicides, in recent years, one of crucial matters is a problem of controlling plant species having acquired resistance to conventional herbicides, such as SU-resistant weeds (weeds acquired resistance to sulfonylurea herbicides). There is a need to develop an active ingredient for herbicides that can control a wide variety of weeds including weeds hard to control such as the SU-resistant weeds, and has an excellent selectivity between crops and weeds.

The present inventors have extensively studied for herbicidal activities of sulfonanilide compounds, and found that sulfonanilide compounds represented by the formula (I) including compounds known before the present application as a part exhibit excellent herbicidal activities and are safe to crops. Therefore they accomplished the present invention.

That is, the present invention provides a herbicide comprising as an active ingredient a sulfonanilide compound represented by the formula (I):

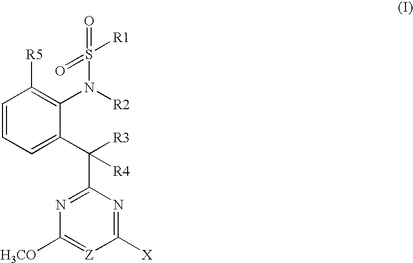

wherein,

R1 represents CHF₂ or CH₂CF₃,

R2 represents hydrogen, C1-3 alkyl, 3-propenyl, or 3-propynyl,

R3 represents hydrogen,

R4 represents hydrogen, hydroxy, or methylthio,

R3 and R4, together with a carbon atom to which they are bonded, may form C═O,

R5 represents halogen or methyl,

X represents methoxy or chlorine, and

Z represents CH or N,

with the proviso that,
(i) when R1 represents CH₂CF₃, R2 represents hydrogen, R5 represents bromine or iodine, X represents methoxy, and Z represents CH,
(ii) when R1 represents CHF₂ and X represents methoxy, R5 represents bromine or iodine, Z represents N, and R2 represents C1-3 alkyl, 3-propenyl, or 3-propynyl,
(iii) when R1 represents CHF₂ and X represents chlorine, Z represents CH.

The sulfonanilide compounds of the formula (I) include known compounds described in Patent Document 7.

Sulfonanilide compounds represented by the following formulae (IA), (IB), and (IC), which are included by the formula (I) of the present invention, are new compounds not described in known publications.

A sulfonanilide compound represented by the formula (IA):

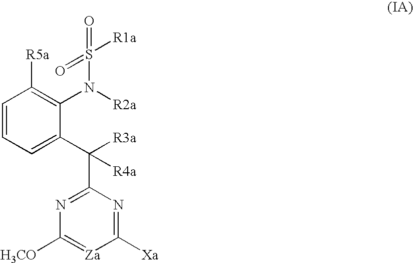

wherein,

R1a represents CH₂CF₃,

R2a represents hydrogen,

R3a represents hydrogen,

R4a represents hydrogen, hydroxy, or methylthio,

R3a and R4a, together with a carbon atom to which they are bonded, may form C═O,

R5a represents bromine or iodine,

Xa represents methoxy, and

Za represents CH.

A sulfonanilide compound represented by the formula (IB):

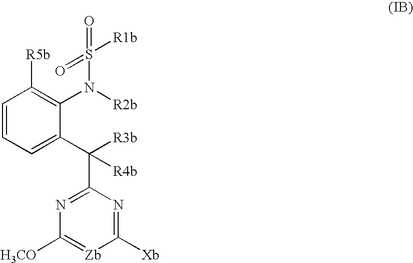

wherein,

R1b represents CHF₂,

R2b represents methyl, ethyl, propyl, 3-propenyl, or 3-propynyl,

R3b represents hydrogen,

R4b represents hydrogen or hydroxy,

R3b and R4b, together with a carbon atom to which they are bonded, may form C═O,

R5b represents bromine or iodine,

Xb represents methoxy, and

Zb represents N.

A sulfonanilide compound represented by the formula (IC):

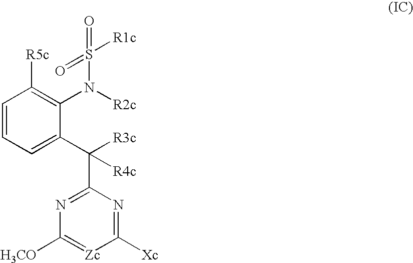

wherein,

R1c represents CHF₂,

R2c represents hydrogen, methyl, ethyl, propyl, 3-propenyl, or 3-propynyl,

R3c represents hydrogen,

R4c represents hydrogen or hydroxy,

R3c and R4c, together with a carbon atom to which they are bonded, may form C═O,

R5c represents fluorine, chlorine, bromine, iodine, or methyl,

Xc represents chlorine, and

Zc represents CH,

with the proviso that,

when R2c represents hydrogen, R5c represents bromine, iodine, or methyl.

The compounds represented by the formulae (IA), (IB), and (IC) can be prepared by, for example, any of the following preparation methods (a) to (h):

preparation method (a): preparation of a compound of the formula (IA) in which R2a represents hydrogen, R3a represents hydrogen, and R4a represents methylthio:

a method of reacting a compound represented by the formula:

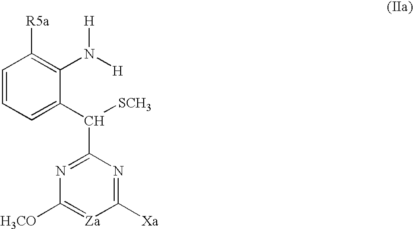

wherein,

R5a, Za and Xa have the same meanings as the aforementioned, with 2,2,2-trifluoroethanesulfonyl chloride.

preparation method (b): preparation of a compound of the formula (IA) in which R2a represents hydrogen, and R3a and R4a, together with a carbon atom to which they are bonded, form C═O:

a method of reacting a compound represented by the formula:

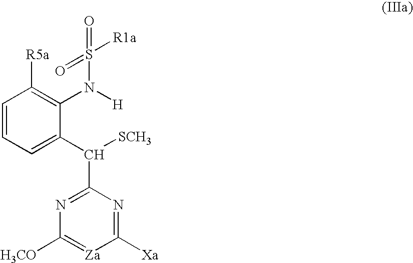

wherein,

R1a, R5a, Xa, and Za have the same meanings as the aforementioned,

in an aqueous hydrogen peroxide solution and acetic acid.

preparation method (c): preparation of a compound of the formula (IC) in which R2c represents hydrogen, R3c and R4c represent hydrogen:

a method of reacting a compound represented by the formula:

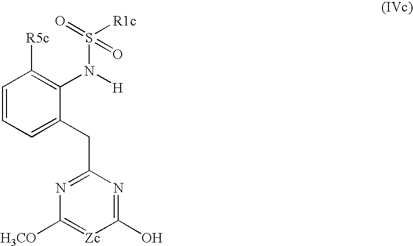

wherein,

R1c, R5c, and Zc have the same meanings as the aforementioned, with a halogenating agent.

preparation method (d): preparation of a compound of the formula (IC) in which R2c represents hydrogen, and R3c and R4c, together with a carbon atom to which they are bonded, form C═O:

a method of reacting a compound represented by the formula:

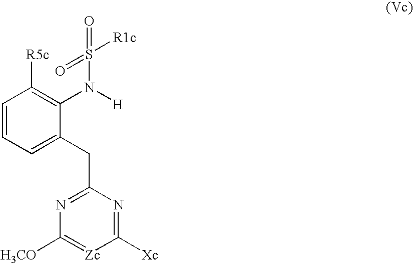

wherein,

R1c, R5c, Xc, and Zc have the same meanings as the aforementioned,

with an oxidizing agent.

preparation method (e): preparation of compounds of the formulae (IB) and (IC) in which R2b or R2c represents methyl, ethyl, propyl, 3-propenyl, or 3-propynyl, and R3b and R4b, or R3c and R4c, together with a carbon atom to which they are bonded, form C═O:

a method of reacting a compound represented by the formula:

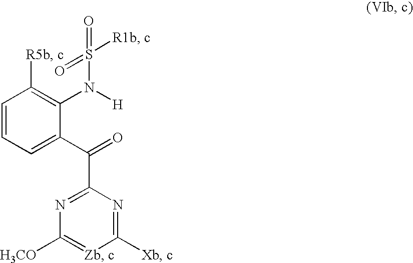

wherein, R1b,c, R5b,c, Xb,c and Zb,c have the same meanings as the aforementioned,
with a compound represented by the formula

R2-L (VII)

wherein, R2 represents methyl, ethyl, propyl, 3-propenyl, or 3-propynyl, and L represents halogen.

preparation method (f): preparation of compounds of the formulae (IA) and (IB) in which R2a represents hydrogen, R2b represents methyl, ethyl, propyl, 3-propenyl, or 3-propynyl, R3a, b represents hydrogen, and R4a,b represents hydroxy:

a method of reacting a compound represented by the formula:

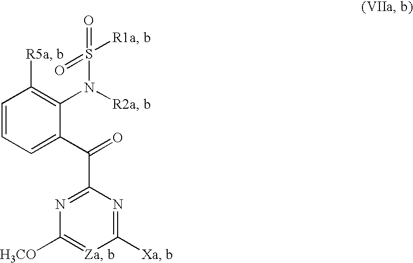

wherein,

R1a,b, R2a,b, R5a,b, Xa,b, and Za,b have the same meanings as the aforementioned,

with an alkaline metal-hydrogen complex compound or a borane complex.

preparation method (g): preparation of a compound of the formula (IC) in which R2c represents hydrogen, R3c represents hydrogen, and R4c represents hydroxy:

a method of reacting a compound represented by the formula:

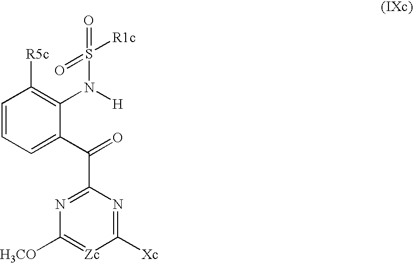

wherein,

R1c, R5c, Xc and Zc have the same meanings as the aforementioned,

with an alkaline metal-hydrogen complex compound or a borane complex.

preparation method (h): preparation of a compound of the formula (IC) in which R2c represents methyl, ethyl, propyl, 3-propenyl, or 3-propynyl, R3c represents hydrogen, and R4c represents hydroxy:

a method of reacting a compound represented by the formula:

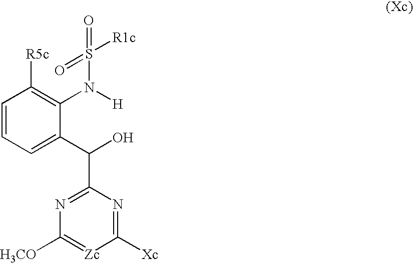

wherein,

R1c, R5c, Xc, and Zc have the same meanings as the aforementioned,

with a compound represented by the formula

R2-L (VII)

wherein, R2 represents methyl, ethyl, propyl, 3-propenyl, or 3-propynyl, and L represents halogen.

The compounds represented by the formula (I) including new compounds of the formulae (IA), (IB), and (IC) exhibit strong herbicide activities.

The sulfonanilide compounds of the formula (I) according to the present invention are conceptionally encompassed by general formulae described in Patent Documents 1 and 2, but compounds of the present invention identified by the formula (I) are not specifically disclosed in Patent Documents 1 and 2. They exhibit substantially excellent herbicide activities, compared with known compounds having analogous structures encompassed by the general formula described in a prior application, Patent Document 6, a part of which compounds is described in Patent Document 6. They also exhibit good herbicide activity to sulfonylurea-resistant weeds.

Compounds of the group described above exhibit good effects as a herbicide for paddy rice directly planted and/or transplanted.

For example, when 2-bromo-6-[(4,6-dimethoxypyrimidin-2-yl)(methylthio)methyl]aniline and 2,2,2-trifluoroethanesulfonyl chloride as raw materials, and pyridine as an acid binder are used, the preparation method (a) can be represented by the following scheme.

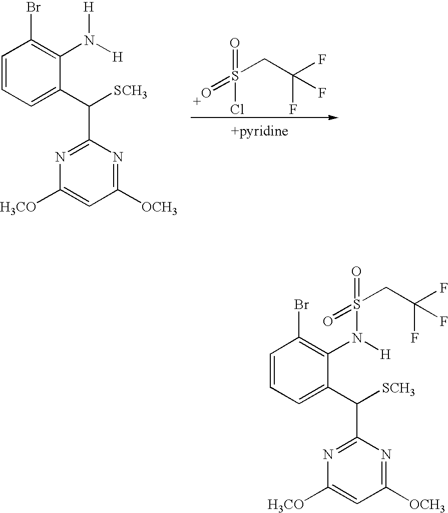

For example, when N-{2-bromo-6-[(4,6-dimethoxypyrimidin-2-yl)(methylthio)methyl]phenyl}-2,2,2-trifluoroethanesulfonamide as a raw material, aqueous hydrogen peroxide solution, and acetic acid are used, the preparation method (b) can be represented by the following scheme.

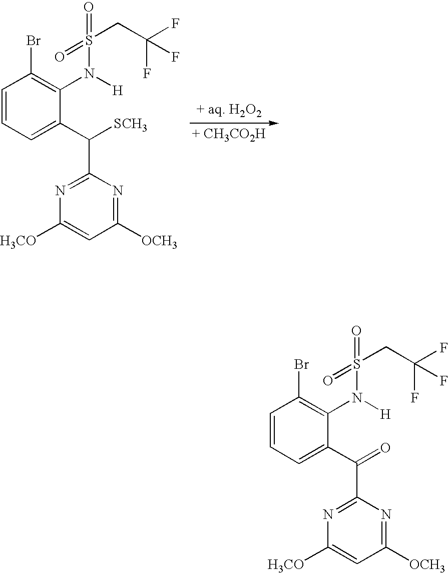

For example, when N-{2-chloro-6-[(4-methoxy-6-hydroxy-1,3-pyrimidin-2-yl)methyl]phenyl}-1,1-difluoromethanesulfonamide as a raw material and phosphorus oxychloride as a halogenating agent are used, the preparation method (c) can be represented by the following scheme.

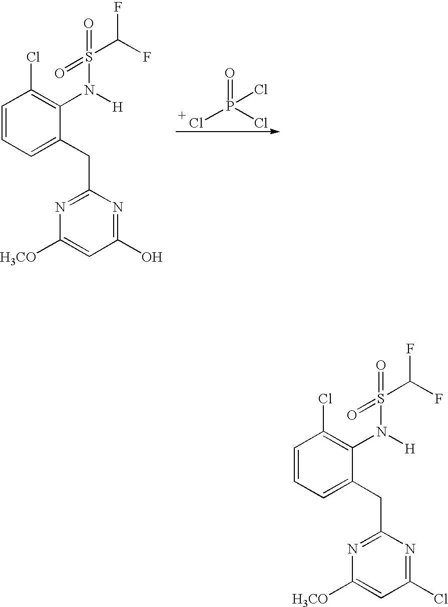

For example, when N-{2-bromo-6-[(4,6-dimethoxytriazin-2-yl)methyl]phenyl}-1,1-difluoromethanesulfonamide as a raw material and chromium(VI) oxide as an oxidizing agent are used, the preparation method (d) can be represented by the following scheme.

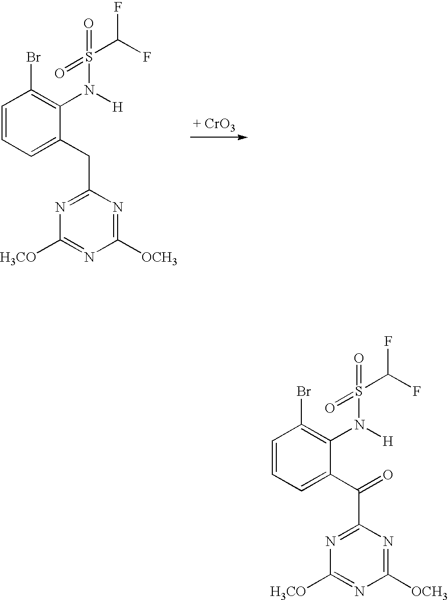

For example, when N-{2-bromo-6-[(4,6-dimethoxy-1,3,5-triazin-2-yl)carbonyl]phenyl}-1,1-difluoromethanesulfonamide as a raw material, methyl iodide, and for example potassium carbonate as an acid binder are used, the preparation method (e) can be represented by the following scheme.

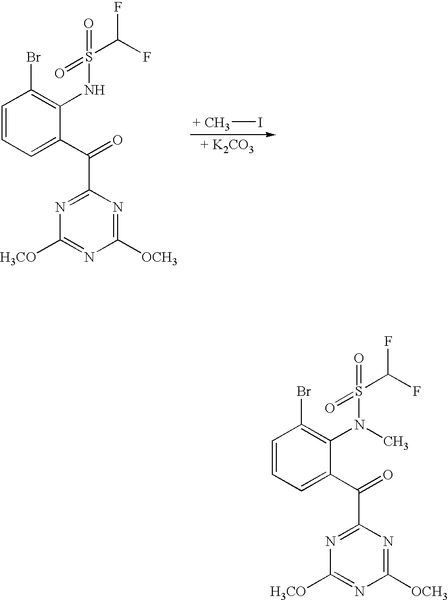

For example, when N-{2-bromo-6-[(4,6-dimethoxypyrimidin-2-yl)carbonyl]phenyl}-2,2,2-trifluoroethanesulfonamide as a raw material and sodium borohydride as a reducing agent are used, the preparation method (f) can be represented by the following scheme.

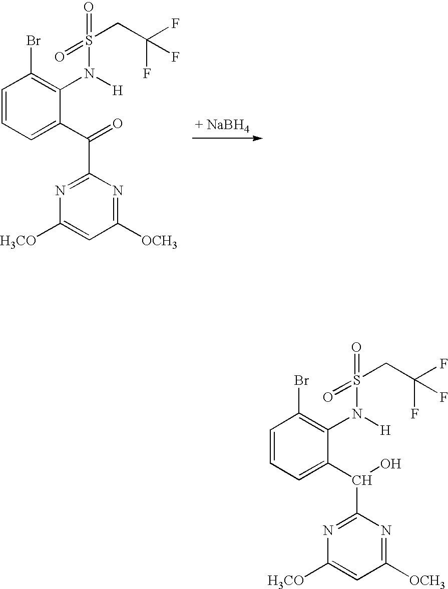

For example, when N-{2-chloro-6-[(4-chloro-6-methoxypyrimidin-2-yl)carbonyl]phenyl}-1,1-difluoromethanesulfonamide as a raw material and sodium borohydride as a reducing agent are used, the preparation method (g) can be represented by the following scheme.

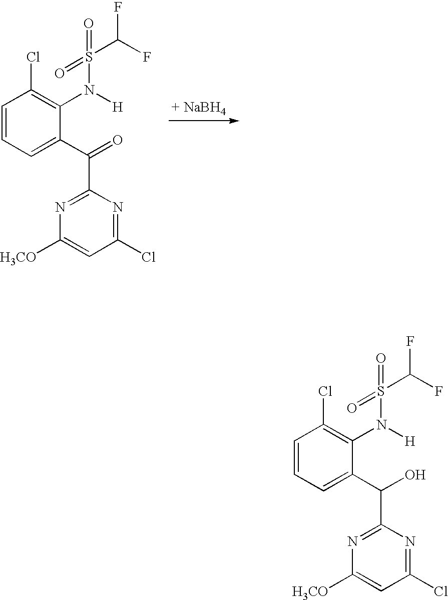

For example, when N-{2-chloro-6-[(4-chloro-6-methoxy-1,3-pyrimidin-2-yl)(hydroxy)methyl]phenyl}-1,1-difluoromethanesulfonamide as a raw material, methyl iodide, and sodium hydrogen carbonate as an acid binder are used, the preparation method (h) can be represented by the following scheme.

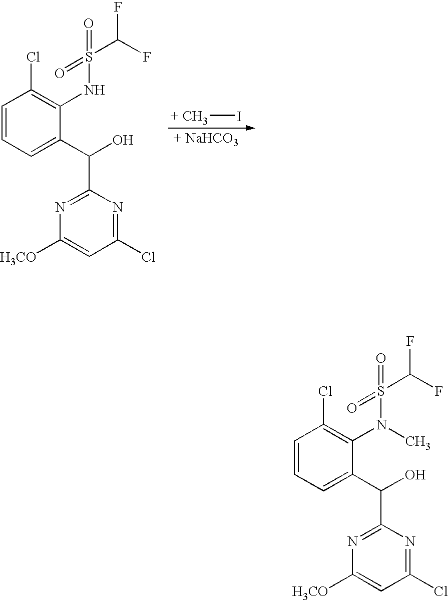

The compound (IIa) used as a raw material in the preparation method (a) can be easily prepared by, for example, reacting a compound represented by the formula:

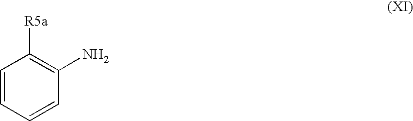

wherein,

R5a has the same meaning as the aforementioned, with 4,6-dimethoxy-2-(methylthiomethyl)pyrimidine in the presence of tert-butyl hypochlorite on the basis of a method described in a pamphlet of PCT WO 96/41799 or the like.

Difluoromethanesulfonyl chloride, trifluoroethanesulfonyl chloride, the compound of the formula (XI), 4,6-dimethoxy-2-(methylthiomethyl)pyrimidine, and 4,6-dimethoxy-2-(methylthiomethyl)triazine are known compounds.

Typical examples of the compound of the formula (IIa) include:
2-bromo-6-[(4,6-dimethoxypyrimidin-2-yl)(methylthio)methyl]aniline and
2-iodo-6-[(4,6-dimethoxypyrimidin-2-yl)(methylthio)methyl]aniline.

In conducting the preparation method (a), the target compound can be prepared by, for example, reacting about 1 mol to 2 mol of 2,2,2-Trifluoroethanesulfonyl chloride with 1 mol of a compound of the formula (IIa) in a diluent such as dichloromethane in the presence of about 1 mol to 5 mol of a base such as pyridine.

The preparation method (a) can be conducted in a substantially wide temperature range.

It is generally conducted at a temperature within the range of about −100° C. to 60° C., and preferably about −80° C. to 40° C.

The reaction may be conducted under a normal temperature, or may be operated under elevated or reduced pressure.

The reaction in the preparation method (a) can be conducted in an appropriate diluent. Examples of the diluent used in the reaction include:

aliphatic, alicyclic, and aromatic hydrocarbons (which may be chlorinated) such as hexane, cyclohexane, ligroin, benzene, toluene, xylene, dichloromethane, chloroform, carbon tetrachloride, 1,2-dichloroethane, chlorobenzene, and dichlorobenzene;

ethers such as ethyl ether, methyl ethyl ether, isopropyl ether, butyl ether, dioxane, dimethoxyethane (DME), tetrahydrofuran (THF), and diethylene glycol dimethyl ether (DGM);

ketones such as acetone, methyl ethyl ketone (MEK), methyl isopropyl ketone, and methyl isobutyl ketone (MBK);

nitriles such as acetonitrile, propionitrile, and acrylonitrile;

esters such as ethyl acetate, and amyl acetate;

acid amides such as dimethylformamide (DMF), dimethylacetamide (DMA), N-methylpyrrolidone, 1,3-dimethyl-2-imidazolidinone, and hexamethylphosphoric triamide (HMPA);

sulfones and sulfoxides such as dimethylsulfoxide (DMSO) and sulfolane; and

bases such as pyridine.

In conducting the preparation method (b), the target compound can be prepared by, for example, reacting about 1 mol to 5 mol of aqueous hydrogen peroxide solution with 1 mol of a compound of the formula (IIIa) in a diluent such as acetic acid.

The reaction in the preparation method (b) can be conducted in an appropriate diluent. Examples of the diluent used in the reaction include:

organic acids such as acetic acid.

The preparation method (b) can be conducted in a substantially wide temperature range.

It is generally conducted at a temperature within the range of about 15° C. to 120° C., and preferably about 15° C. to 100° C.

The reaction may be conducted under a normal temperature, or may be operated under elevated or reduced pressure.

The reaction in the preparation method (b) can be conducted in an appropriate diluent. Examples of the diluent used in the reaction include:

water;

aliphatic, alicyclic, and aromatic hydrocarbons (which may be chlorinated) such as hexane, cyclohexane, ligroin, benzene, toluene, xylene, dichloromethane, chloroform, carbon tetrachloride, 1,2-dichloroethane, chlorobenzene, and dichlorobenzene;

nitriles such as acetonitrile, propionitrile, and acrylonitrile;

esters such as ethyl acetate and amyl acetate;

acid amides such as dimethylformamide (DMF), dimethylacetamide (DMA), N-methylpyrrolidone, 1,3-dimethyl-2-imidazolidinone, and hexamethylphosphoric triamide (HMPA);

sulfones and sulfoxides such as dimethylsulfoxide (DMSO) and sulfolane;

organic acids such as formic acid, acetic acid, trifluoroacetic acid, and propionic acid; and

bases such as pyridine.

The compound (IVc) used as a raw material in the preparation method (c) can be prepared by, for example, reacting a compound represented by the formula:

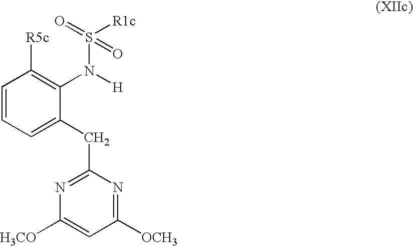

wherein,

R1c and R5c have the same meanings as the aforementioned, with hydrobromic acid according to a method described in Journal of Heterocyclic Chemistry 26 913-915 (1989) or the like.

The compound of the formula (XIIc) can be prepared in the same manner as a method described in Patent Document 5.

Typical examples of the compound of the formula (IVc) include:
N-{2-fluoro-6-[(4-hydroxy-6-methoxy-pyrimidin-2-yl)methyl]phenyl}-1,1-difluoromethanesulfonamide,
N-{2-chloro-6-[(4-hydroxy-6-methoxy-pyrimidin-2-yl)methyl]phenyl}-1,1-difluoromethanesulfonamide,
N-{2-bromo-6-[(4-hydroxy-6-methoxy-pyrimidin-2-yl)methyl]phenyl}-1,1-difluoromethanesulfonamide,
N-{2-iodo-6-[(4-hydroxy-6-methoxy-pyrimidin-2-yl)methyl]phenyl}-1,1-difluoromethanesulfonamide, and
N-{2-methyl-6-[(4-hydroxy-6-methoxy-pyrimidin-2-yl)methyl]phenyl}-1,1-difluoromethanesulfonamide.

Typical examples of the compound of the formula (XIIc) include:
N-{2-fluoro-6-[(4,6-dimethoxy-pyrimidin-2-yl)methyl]phenyl}-1,1-difluoromethanesulfonamide,
N-{2-chloro-6-[(4,6-dimethoxy-pyrimidin-2-yl)methyl]phenyl}-1,1-difluoromethanesulfonamide,
N-{2-bromo-6-[(4,6-dimethoxy-pyrimidin-2-yl)methyl]phenyl}-1,1-difluoromethanesulfonamide,
N-{2-iodo-6-[(4,6-dimethoxy-pyrimidin-2-yl)methyl]phenyl}-1,1-difluoromethanesulfonamide, and
N-{2-methyl-6-[(4,6-dimethoxy-pyrimidin-2-yl)methyl]phenyl}-1,1-difluoromethanesulfonamide.

In conducting the preparation method (c), the target compound can be prepared by, for example, reacting about 10 mol to 20 mol of a halogenating agent such as phosphorus oxychloride with 1 mol of a compound of the formula (IVc) in the presence of 1 mol of N,N-dimethylaniline.

The preparation method (c) can be conducted in a substantially wide temperature range.

It is generally conducted at a temperature within the range of about 0° C. to 180° C., and preferably about 20° C. to 120° C.

The reaction may be conducted under a normal temperature, or may be operated under elevated or reduced pressure.

The reaction in the preparation method (c) can be conducted in an appropriate diluent. Examples of the diluent used in the reaction include:

aliphatic, alicyclic, and aromatic hydrocarbons (which may be chlorinated) such as hexane, cyclohexane, ligroin, benzene, toluene, xylene, dichloromethane, chloroform, carbon tetrachloride, 1,2-dichloroethane, chlorobenzene, and dichlorobenzene;

ethers such as ethyl ether, methyl ethyl ether, isopropyl ether, butyl ether, dioxane, dimethoxyethane (DME), tetrahydrofuran (THF), and diethylene glycol dimethyl ether (DGM);

ketones such as acetone, methyl ethyl ketone (MEK), methyl isopropyl ketone, and methyl isobutyl ketone (MIBK);

nitriles such as acetonitrile, propionitrile, and acrylonitrile;

acid amides such as dimethylformamide (DMF), dimethylacetamide (DMA), N-methylpyrrolidone, 1,3-dimethyl-2-imidazolidinone, and hexamethylphosphoric triamide (HMPA); and

sulfones and sulfoxides such as dimethylsulfoxide (DMSO), and sulfolane.

In conducting the preparation method (d), the target compound can be prepared by, for example, reacting about 1 mol to 10 mol of chromium(VI) oxide with 1 mol of a compound of the formula (Vc) in a diluent such as acetic acid.

Examples of the oxidizing agent used in the preparation method (d) include chromium(VI) oxide, manganese dioxide, and selenium dioxide.

The preparation method (d) can be conducted in the presence of acid. Examples of the acid catalyst include mineral acids such as hydrochloric acid, sulfuric acid, nitric acid, hydrobromic acid, and sodium hydrogen sulfite; and organic acids such as formic acid, acetic acid, trifluoroacetic acid, propionic acid, methanesulfonic acid, benzenesulfonic acid, and p-toluenesulfonic acid.

The preparation method (d) can be conducted in a substantially wide temperature range. It is generally conducted at a temperature within the range of about −100° C. to 150° C., and preferably about 20° C. to 120° C.

The reaction is desirably conducted under a normal temperature, but may be operated under elevated or reduced pressure.

The compound of the formula (VIb) used as a raw material in the preparation method (e) can be prepared in the same manner as the preparation method (d).

Typical examples of the compound of the formula (VIb) used as a raw material in the preparation method (e) include:
N-{2-bromo-6-[(4,6-dimethoxy-1,3,5-triazin-2-yl)carbonyl]phenyl}-1,1-difluoromethanesulfonamide, and
N-{2-iodo-6-[(4,6-dimethoxy-1,3,5-triazin-2-yl)carbonyl]phenyl}-1,1-difluoromethanesulfonamide.

The compound of the formula (VII) reacted with the compound of the formula (VIb,c) in the preparation method (e) is a known compound per se. Typical examples of the compound include:

methyl iodide, ethyl iodide, n-propyl iodide, 3-bromopropene, and propargyl bromide.

In the preparation method (e), compounds of the formulae (IB) and (IC) can be prepared by reacting about 2 mol to 5 mol of the compound of the formula (VII) with 1 mol of a compound of the formula (VIb,c) in a diluent such as acetonitrile in the presence of about 2 mol to 5 mol an acid binder.

The preparation method (e) can be conducted in the presence of an acid binder. Examples of the acid binder include: inorganic bases such as hydrides, hydroxides, carbonates, and bicarbonates of alkaline metals and alkaline earth metals

including sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide;

and inorganic alkaline metal amides including lithium amide, sodium amide, and potassium amide; and

organic bases such as alcoholates, tertiary amines, dialkylaminoanilines and pyridines including triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO), and 1,8-diazabicyclo[5,4,0]undec-7-ene (DAU);

organolithium compounds including methyllithium, n-butyllithium, sec-butyllithium, tert-butyllithium, phenyllithium, dimethylcopperlithium, lithium diisopropyl amide, lithium cyclohexylisopropyl amide, lithium dicyclohexyl amide, n-butyllithium+DABCO, n-butyllithium+DBU, and n-butyllithium+TMEDA.

The preparation method (e) can be conducted in a substantially wide temperature range. It is generally conducted at a temperature within the range of about −100° C. to 130° C., and preferably about −80° C. to 130° C. The reaction is desirably conducted under a normal temperature, but may be operated under elevated or reduced pressure.

The reaction in the preparation method (e) can be conducted in an appropriate diluent. Examples of the diluent used in the reaction include:

aliphatic, alicyclic, and aromatic hydrocarbons (which may be chlorinated) such as hexane, cyclohexane, ligroin, benzene, toluene, xylene, dichloromethane, chloroform, carbon tetrachloride, 1,2-dichloroethane, chlorobenzene, and dichlorobenzene;

ethers such as ethyl ether, methyl ethyl ether, isopropyl ether, butyl ether, dioxane, dimethoxyethane (DME), tetrahydrofuran (THF), and diethylene glycol dimethyl ether (DGM);

ketones such as acetone, methyl ethyl ketone (MEK), methyl isopropyl ketone, and methyl isobutyl ketone (MIBK);

nitriles such as acetonitrile, propionitrile, and acrylonitrile;

esters such as ethyl acetate and amyl acetate;

acid amides such as dimethylformamide (DMF), dimethylacetamide (DMA), N-methylpyrrolidone, 1,3-dimethyl-2-imidazolidinone, and hexamethylphosphoric triamide (HMPA);

sulfones and sulfoxides such as dimethylsulfoxide (DMSO) and sulfolane; and

bases such as pyridine.

In conducting the preparation methods (f) and (g), the target compounds can be prepared by, for example, reacting about 0.25 mol to 2 mol of sodium borohydride with 1 mol of a compound of the formula (VIIIa,b) or (IXc) in a diluent such as methanol.

Examples of the alkaline metal-hydrogen complex compound and the borane complex used in the preparation methods (f) and (g) include sodium borohydride, lithium aluminum hydride, dimethylsulfide borane, and pyridine-borane.

The preparation methods (f) and (g) can be conducted in a substantially wide temperature range. It is generally conducted at a temperature within the range of about −100° C. to 60° C., and preferably about −80° C. to 40° C. The reaction is desirably conducted under a normal temperature, but may be operated under elevated or reduced pressure.

The reactions in the preparation methods (f) and (g) can be conducted in an appropriate diluent. Examples of the diluent used in the reactions include:

aliphatic, alicyclic, and aromatic hydrocarbons (which may be chlorinated) such as pentane, hexane, cyclohexane, petroleum ether, ligroin, benzene, toluene, xylene, dichloromethane, chloroform, carbon tetrachloride, 1,2-dichloroethane, chlorobenzene, and dichlorobenzene;

ethers such as ethyl ether, methyl ethyl ether, isopropyl ether, butyl ether, dioxane, dimethoxyethane (DME), tetrahydrofuran (THF), and diethylene glycol dimethyl ether (DGM);

nitriles such as acetonitrile and propionitrile;

alcohols such as methanol, ethanol, isopropanol, butanol, and ethylene glycol;

esters such as ethyl acetate and amyl acetate;

acid amides such as dimethylformamide (DMF), dimethylacetamide (DMA), N-methylpyrrolidone, 1,3-dimethyl-2-imidazolidinone, and hexamethylphosphoric triamide (HMPA);

sulfones and sulfoxides such as dimethylsulfoxide (DMSO) and sulfolane; and

bases such as pyridine.

In conducting the preparation method (h), the target compound can be prepared by, for example, reacting about 1 mol to 5 mol a compound of the formula (VII) with 1 mol of a compound of the formula (Xc) in a diluent such as acetonitrile in the presence of an acid binder such as sodium hydrogen carbonate.

The compound of the formula (VII) reacted with the compound of the formula (Xc) in the preparation method (h) has the same meaning as the formula (VII) in the preparation method (e).

The preparation method (h) can be conducted in the presence of the acid binder. Examples of the acid binder include: inorganic bases such as hydrides, hydroxides, carbonates, and bicarbonates of alkaline metals and alkaline earth metals including

sodium hydride, lithium hydride, sodium hydrogen carbonate, potassium hydrogen carbonate, sodium carbonate, potassium carbonate, lithium hydroxide, sodium hydroxide, potassium hydroxide, and calcium hydroxide;

and inorganic alkaline metal amides including as lithium amide, sodium amide, and potassium amide; and

organic bases such as alcoholates, tertiary amines, dialkylaminoanilines and pyridines including triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO), and 1,8-diazabicyclo[5,4,0]undec-7-ene (DAU); and

organolithium compounds including methyllithium, n-butyllithium, sec-butyllithium, tert-butyllithium, phenyllithium, dimethylcopperlithium, lithium diisopropyl amide, lithium cyclohexylisopropyl amide, lithium dicyclohexyl amide, n-butyllithium+DABCO, n-butyllithium+DBU, and n-butyllithium+TMEDA. The acid binder is desirably sodium hydrogen carbonate.

The preparation method (h) can be conducted in a substantially wide temperature range. It is generally conducted at a temperature within the range of about −100° C. to 130° C., and preferably about −80° C. to 100° C. The reaction is desirably conducted under a normal temperature, but may be operated under elevated or reduced pressure.

The reaction in the preparation method (h) can be conducted in an appropriate diluent. Examples of the diluent used in the reaction include:

aliphatic, alicyclic, and aromatic hydrocarbons (which may be chlorinated) such as hexane, cyclohexane, ligroin, toluene, xylene, dichloromethane, chloroform, carbon tetrachloride, 1,2-dichloroethane, and chlorobenzene;

ethers such as ethyl ether, methyl ethyl ether, isopropyl ether, butyl ether, dioxane, dimethoxyethane (DME), tetrahydrofuran (THF), and diethylene glycol dimethyl ether (DGM);

ketones such as acetone, methyl ethyl ketone (MEK), methyl isopropyl ketone, and methyl isobutyl ketone (MIBK);

nitriles such as acetonitrile, propionitrile, and acrylonitrile;

esters such as ethyl acetate and amyl acetate;

acid amides such as dimethylformamide (DMF), dimethylacetamide (DMA), N-methylpyrrolidone, 1,3-dimethyl-2-imidazolidinone, and hexamethylphosphoric triamide (HMPA);

sulfones and sulfoxides such as dimethylsulfoxide (DMSO) and sulfolane; and

bases such as pyridine.

The diluent is desirably acetonitrile.

The active compounds of the formula (I) of the present invention exhibit excellent herbicide activity to various kinds of weeds and can be used as herbicides, as will be described in Biological Test Examples described later. In the present specification, the weeds mean, in a broad sense, all plants growing in locations where they are undesired. The compounds of the present invention act as a selective herbicide depending on the concentration thereof at the time of use. For example, the active compounds of the present invention can be used against the following weeds grown among the following cultivated plants.

The genus of dicotyledonous weeds: Sinapis, Capsella, Leipidium, Galium, Stellaria, Chenopodium, Kochia, Urtica, Senecio, Amaranthus, Portulaca, Xanthium, Ipomoea, Polygonum, Ambrosia, Cirsium, Sonchus, Solanum, Rorippa, Lamium, Veronica, Datura, Viola, Galeopsis, Papaver, Centaurea, Galinsoga, Rotala, Lindernia, SeSbania, Trifolium, Abutilon, Lamium, Matricaria, Artemisia, Sesbania, Pharbitis and the like.

The genus of dicotyledonous cultivated plants: Gossypium, Glycine, Beta, Daucus, Phaseolus, Pisum, Solanum, Linum, Ipomoea, Vicia, Nicotiana, Lycopersicon, Arachis, Brassica, Lactuca, Cucumis, Cucurbita and the like.

The genus of monocotyledonous weeds: Echinochlona, Setaria, Panicum, Digitaria, Phleum, Poa, Festuca, Eleusine, Lolium, Bromus, Avena, Cyperus, Sorghum, Agropyron, Monochoria, Fimbristylis, Sagittaria, Eleocharis, Scirpus, Paspalum, Ischaemum, Agrostis, Alopecurus, Cynodon, Commelina, Brachiaria, Leptochloa and the like.

The genus of monocotyledonous cultivated plants: Oryza, Zea, Triticum, Hordeum, Avena, Secale, Sorghum, Panicum, Saccharum, Ananas, Asparagus, Allium and the like.

The active compounds of the formula (I) of the present invention can be used for weeds in paddy fields. Examples of the weeds in paddy fields to be controlled by the active compounds of the present invention include:

Dicotyledonous plants of the following genera: Polygonum, Rorippa, Rotala, Lindernia, Bidens, Dopatrium, Eclipta, Elatine, Gratiola, Lindernia, Ludwigia, Oenanthe, Ranunculus, Deinostema, and the like.

Monocotyledonous plants of the following genera: Echinochloa, Panicum, Poa, Cyperus, Monochoria, Fimbristylis, Sagittaria, Eleocharis, Scirpus, Alisma, Aneilema, Blyxa, Eriocaulon, Potamogeton, Brachiaria, Leptochloa, Spbenoclea, and the like.

More specifically, the active compounds of the formula (I) of the present invention can be used for the following representative weeds in paddy fields.

TABLE 1
Botanical name

Dicotyledonous plants
Rotala indica Koehne
Lindernia procumbens Philcox
Lindernia dubia L. Penn.
Lindernia angustifolia
Ludwigia prostrata Roxburgh
Potamogeton distinctus A. Benn
Elatine triandra Schk
Oenanthe javanica
Monocotyledonous plants
Echinochloa oryzicola Vasing
Eleocharis acicularis L.
Eleocharis kuroguwai Ohwi
Cyperus difformis L.
Cyperus serotinus Rottboel
Scirpus juncoides Roxburgh
Monochoria vaginalis Presl
Sagittaria pygmaea Miq
Alisma canaliculatum A. Br. et Bouche
Sagittaria trifolia
Monochoria korsakowii
Brachiaria plantaginea
Leptochloa chinensis

The active compounds of the formula (I) of the present invention can be effectively used for weeds resistant against sulfonylurea herbicides. Examples of the weeds include those described above.

The active compounds of the formula (I) of the present invention are not particularly limited for use to these grass weeds but are similarly applicable to other grass weeds.

Further, the active compounds of the present invention can be used for controlling weeds in cultivation of perennial plants. For example, the active compounds of the present invention can be used for forestation, forestation for decorative plants, orchards, vineyards, citrus orchards, nuts orchards, banana cultivation farms, coffee plantations, tea plantations, rubber plantations, oil palm plantations, cocoa plantations, small orchards, hop cultivation farms, and the like. The active compounds of the present invention can also be used for selectively controlling weeds in cultivation of annual plants.

The active compounds of the present invention can be formulated in a conventional formulation for practical use. Examples of the formulation form include solutions, wettable powders, emulsions, suspensions, dusts, water-dispersible granules, tablets, granules, suspended emulsion concentrates, microcapsules in a polymer substance, and jumbo formulation-package.

These formulations can be prepared by conventionally known methods per se, for example, by mixing an active compound with a developer, i.e., a liquid or solid diluent or carrier, and if necessary, together with a surfactant, i.e., an emulsifier and/or a dispersant and/or a foaming agent.

Examples of the liquid diluent or carrier include aromatic hydrocarbons (e.g., xylene, toluene, and alkylnaphthalene), chlorinated aromatic or chlorinated aliphatic hydrocarbons (e.g., chlorobenzenes, ethylene chlorides, and methylene chloride), aliphatic hydrocarbons [e.g., paraffins (e.g., mineral oil fractions) such as cyclohexane], alcohols (e.g., butanol and glycol), and ethers, esters, and ketones thereof (e.g., acetone, methyl ethyl ketone, methyl isobutyl ketone, and cyclohexanone), strongly polar solvents (e.g., dimethylformamide and dimethyl sulfoxide) and water. When water is used as a developer, an organic solvent may be used as an auxiliary solvent.

Examples of the solid diluent or carrier include pulverized natural minerals (e.g., kaolin, clay, talc, chalk, quartz, attapulgite, montmorillonite, and diatomaceous earth), pulverized synthetic minerals (e.g., highly dispersed silicic acid, alumina, and silicates). Examples of the solid carrier for granules include pulverized and classified rocks (e.g., calcite, marble, pumice, meerschaum, and dolomite), synthesized inorganic and organic particles, fine particles of organic substances (e.g., sawdust, husks of coconuts, stems of Sorghum, and stalks of tobacco).

Examples of the emulsifying agent and/or foaming agent include nonionic and anionic emulsifying agents [e.g., polyoxyethylene fatty acid ester, polyoxyethylene fatty acid alcohol ether (e.g., alkyl aryl polyglycol ethers, alkylsulfonates, alkylsulfates, and arylsulfonates)], and hydrolysis products of albumin.

Examples of the decomposition agent include lignin sulfite waste solution and methyl cellulose.

A fixing agent may be used for the formulation (dusts, granules, and emulsions) and examples thereof include carboxymethyl cellulose, natural and synthetic polymers (e.g., gum arabic, polyvinyl alcohol, and polyvinyl acetate).

A coloring agent may also be used and examples thereof include inorganic pigments (e.g., iron oxide, titanium oxide, and Prussian blue); organic dyes such as alizarin dyes, azo dyes, and metal phthalocyanine dyes; and a trace element such as metal salts of iron, manganese, boron, copper, cobalt, molybdenum, and zinc.

The formulation contains the active compounds of the formula (I) generally in a range of 0.01 to 95% by weight and preferably in a range of 0.1 to 90% by weight.

The active compounds of the formula (I) of the present invention can be used for controlling weeds as it is or in a formulation form. The active compounds of the formula (I) of the present invention may also be used in combination with a known herbicide. A mixed herbicide composition with a known herbicide may be prepared previously in a final formulation or may be prepared by tank-mixing at the time of use. Specific examples of the herbicide usable in combination with the compounds of the formula (I) of the present invention in the mixed herbicide composition include following herbicides, which are described as common names.

Acetamide herbicides: pretilachlor, butachlor, tenylchlor, and alachlor, etc.;

Amide herbicides: clomepropand etobenzanide, etc.;

Benzofuran herbicides: benfuresate, etc.;

Indandione herbicides: indanofan, etc.;

Pyrazole herbicides: pyrazolate, benzofenap, and pyrazoxyfen, etc.;

Oxazinone herbicides: oxaziclomefone, etc.;

Sulfonylurea herbicides: bensulfuron methyl, azimsulfron, imazosulfuron, pyrazosulfuron ethyl, cyclosulfamuron, ethoxysulfuron, and halosulfuron methyl, etc.;

Thiocarbamate herbicides: thiobencarb, molinate, and pyributicarb, etc.;

Triazolopyrimidine herbicides: penoxsulam, flumetsulam, florasulam, etc.;

Triazine herbicides: dimethametryn and simetryn, etc.;

Triazole herbicides: cafenstrole, etc.;

Quinoline herbicides: quinclorac, etc.;

Isoxazole herbicides: isoxaflutole, etc.;

Dithiophosphate herbicides: anilofos, etc.;

Oxyacetamide herbicides: mefenacet and flufenacet, etc.;

Tetrazolinone herbicides: fentrazamide, etc.;

Dicarboxylmide herbicides: pentoxazone, etc.;

Oxadiazolone herbicides: oxadiargyl and oxadiazon, etc.;

Trione herbicides: sulcotrione and benzobicyclon, etc.;

Phenoxypropionate herbicides: cyhalofop butyl, etc.;

Benzoic acid herbicides: pyriminobac methyl and bispyribac sodium, etc.;

Diphenyl ether herbicides: chlomethoxynil and oxyfluorfen, etc.;

Pyridine dicarbothioate herbicides: dithiopyr, etc.;

Phenoxy herbicides: MCPA and MCPB, etc.;

Urea herbicides: daimuron and cumyluron, etc.;

Naphthalenedione herbicides: quinoclamin, etc.;

Isoxazolidinone herbicides: clomazone, etc.;

Imidazolinone herbicides: imazethapyr and imazamox, etc.

These active compounds are known herbicides described in “Pesticide Manual”, British Crop Protect Council (2000).

The active compounds of the formula (I) of the present invention mixed with a herbicide safener may be provided with a wider range spectrum controlling weeds and a wider range of applicability as a selective herbicide with lessened herbicide damage.

Examples of the herbicide safener include the following compounds named as the common names or development codes:

AD-67, BAS-145138, benoxacor, chloquintocet-mexyl, cyometrinil, 2,4-D, DKA-24, dichlormid, dimuron, fenchlorim, fenchlorazole-ethyl, flurazole, fluxofenim, furilazole, isoxadifen-ethyl, mefenpyr-diethyl, MG-191, naphthalic anhydride, oxabetrinil, PPG-1292, and R-29148.

These herbicide safeners are also disclosed in “Pesticide Manual”, British Crop Protect Council (2000).

The mixed herbicide composition containing the compounds of the formula (I) of the present invention and the above herbicides may further be mixed with the above herbicide safeners. The mixing lessens the herbicide damage and provides the composition with a wider range spectrum in controlling weeds and a wider range of applicability as a selective herbicide.

Surprisingly, some herbicide mixture compositions containing the compound of the present invention and a known herbicide and/or a herbicide safener exhibit synergetic effects.

The active compounds of the formula (I) of the present invention can be used directly as it is or in the form of a formulation such as prepared liquids for spraying, emulsions, tablets, suspensions, dusts, pastes, or granules or in the form of a further diluted formulation thereof. The active compounds of the present invention can be applied in a manner of watering, spraying, atomizing, spreading granules, or the like.

The active compounds of the formula (I) of the invention can be used in any stage before or after germination of plants and can be added into soil before seeding.

The application amount of the active compounds of the invention can be varied in a practically applicable range and basically differs depending on the desired effects. In the case where the compound is used as a herbicide, the application dose is, for example, about 0.0001 to about 4 kg, preferably about 0.001 to about 1 kg of active compound per hectare.

### EXAMPLES

Preparation and use of the compounds of the invention will be described by way of the following Examples. However, the present invention is not intended to be limited to these Examples.

### Synthesis Example 1

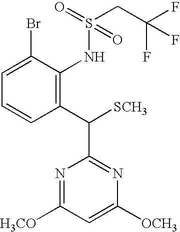

0.45 g of 2-bromo-6-[(4,6-dimethoxypyrimidin-2-yl)(methylthio)methyl]aniline (1.22 mmol) was dissolved in dichloromethane (3 ml) and 0.08 g of pyridine (0.97 mmol) was added to the resultant solution, and the resulting solution was cooled to −5° C. 0.18 g of 2,2,2-trifluoroethanesulfonyl chloride (0.97 mmol) was added to the solution. The resulting reaction mixture was stirred for two days at room temperature, water was added to the mixture, and the product was extracted with dichloromethane three times. The organic layer was washed with water and dried. Dichloromethane was evaporated to give an oily residue. The residue was purified by column chromatography to give 0.36 g of desired product, 2-bromo-6-[(4,6-dimethoxypyrimidin-2-yl)methylthiomethyl]-N-2,2,2-trifluoroethanesulfonanilide (yield: 57%).

¹H NMR (CDCl₃, 300 MHz) δ 2.04 (3H, s), 3.94 (6H, s), 4.08-4.29 (1H, m), 4.61-4.75 (1H, m), 5.81 (1H, s), 5.90 (1H, s), 7.24 (1H, t), 7.56 (1H, dd), 8.06 (1H, dd), 9.16 (1H, br)

### Synthesis Example 2

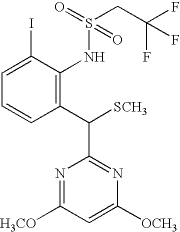

1.00 g of 2-iodo-6-[(4,6-dimethoxypyrimidin-2-yl)(methylthio)methyl]aniline (2.40 mmol) was dissolved in dichloromethane (3 ml), and 0.19 g of pyridine (2.40 mmol) was added to the resultant solution, and the resulting solution was cooled to −5° C. 0.44 g of 2,2,2-trifluoroethanesulfonyl chloride (2.40 mmol) was added to the solution. The reaction mixture was stirred for two days at room temperature, water was added to the mixture, and the product was extracted with dichloromethane three times. The organic layer was washed with water and dried. Dichloromethane was evaporated to give an oily residue. The residue was purified by column chromatography to give 1.20 g of desired product, 2-iodo-6-[(4,6-dimethoxypyrimidin-2-yl)(methylthio)methyl]-N-2,2,2-trifluoroethanesulfonanilide (yield: 89%).

¹H NMR (CDCl3, 300 MHz) δ 2.04 (3H, s), 3.94 (6H, s), 4.23-4.31 (1H, m), 4.80-4.87 (1H, m), 5.89 (1H, s), 5.90 (1H, s), 7.08 (1H, t), 7.82 (1H, dd), 8.09 (1H, dd), 9.14 (1H, br)

### Synthesis Example 3

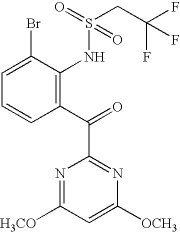

0.25 g of 2-bromo-6-[(4,6-dimethoxypyrimidin-2-yl)(methylthio)methyl]-N-2,2,2-trifluoroethanesulfonanilide (0.48 mmol) was diluted with acetic acid (5 ml), 33% aqueous hydrogen peroxide solution at room temperature was stirred overnight at room temperature and then stirred for two hours at 80° C. The reaction mixture was cooled to room temperature, diluted with water, and the product was extracted with ethyl acetate three times. The organic layer was washed with water and dried. Ethyl acetate was evaporated to give an oily residue. The residue was purified by column chromatography to give 0.2 g of desired product, 2-bromo-6-[(4,6-dimethoxypyrimidin-2-yl)carbonyl]-N-2,2,2-trifluoroethanesulfonanilide (yield: 85%).

¹H NMR (CDCl3, 300 MHz) 63.94 (6H, s), 4.06 (2H, q), 6.18 (1H, s), 7.32 (1H, t), 7.60 (1H, br), 7.69 (1H, dd), 7.88 (1H, dd)

### Synthesis Example 4

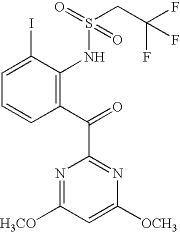

1.00 g of 2-iodo-6-[(4,6-dimethoxypyrimidin-2-yl)(methylthio)methyl]-N-2,2,2-trifluoroethanesulfonanilide (1.78 mmol) was diluted with acetic acid (8 ml), 33% aqueous hydrogen peroxide solution at room temperature was stirred overnight at room temperature and then stirred for two hours at 80° C. The reaction mixture was cooled to room temperature, diluted with water, and the product was extracted with ethyl acetate three times. The organic layer was washed with water and dried. Ethyl acetate was evaporated to give an oily residue. The residue was purified by column chromatography to give 0.55 g of desired product, 2-iodo-6-[(4,6-dimethoxypyrimidin-2-yl)carbonyl]-N-2,2,2-trifluoroethanesulfonanilide (yield: 58%).

¹H NMR (CDCl3, 300 MHz) δ3.93 (6H, s), 4.06 (2H, q), 6.18 (1H, s), 7.17 (1H, t), 7.59 (1H, br), 7.68 (1H, dd), 8.10 (1H, dd)

### Synthesis Example 5

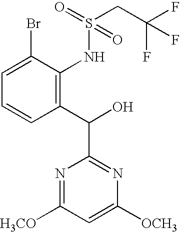

0.12 g of 2-bromo-6-[(4,6-dimethoxypyrimidin-2-yl)carbonyl]-N-2,2,2-trifluoroethanesulfonanilide (0.25 mmol) was dissolved in methanol (5 ml) and the resultant solution was cooled to 5° C., and sodium borohydride was added to the solution, which was being stirred. The resultant mixture was stirred for two hours at room temperature. The reaction liquid was evaporated under a reduced pressure and the residue was neutralized with citric acid. The organic layer was separated and collected. The aqueous layer was further extracted with ethyl acetate three times. The organic layers were washed with water, dried, and the solvent was evaporated under a reduced pressure to give 0.12 g of desired product, 2-bromo-6-[(4,6-dimethoxypyrimidin-2-yl)(hydroxy)methyl]-N-2,2,2-trifluoroethanesulfonanilide (yield: 96%).

¹H NMR (CDCl3, 300 MHz) δ 3.97 (6H, s), 4.10-4.24 (1H, m), 4.65-4.79 (1H, m), 5.00 (1H, s), 5.98 (1H, m), 6.30 (1H, d), 7.20 (1H, t), 7.58 (1H, dd), 7.68 (1H, dd), 10.01 (1H, br)

### Synthesis Example 6

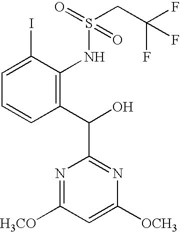

0.40 g of 2-iodo-6-[(4,6-dimethoxypyrimidin-2-yl)carbonyl]-N-2,2,2-trifluoroethanesulfonanilide (0.75 mmol) was dissolved in methanol (10 ml) and the resultant solution was cooled to 5° C., and 0.06 g of sodium borohydride (1.51 mmol) was added to the solution, which was being stirred. The resultant mixture was stirred for two hours at room temperature. The reaction liquid was evaporated under a reduced pressure and the residue was neutralized with citric acid. The organic layer was separated and collected. The aqueous layer was further extracted with ethyl acetate three times. The organic layers were washed with water, dried, and the solvent was evaporated under a reduced pressure to give 0.37 g of desired product, 2-iodo-6-[(4,6-dimethoxypyrimidin-2-yl)(hydroxy)methyl]-N-2,2,2-trifluoroethanesulfonanilide (yield: 93%).

¹H NMR (CDCl3, 300 MHz) δ 3.97 (6H, s), 4.20-4.30 (1H, m), 4.79-4.93 (1H, m), 5.00 (1H, s), 5.97 (1H, m), 6.36 (1H, d), 7.07 (1H, t), 7.69 (1H, dd), 7.83 (1H, dd), 10.00 (1H, br)

### Synthesis Example 7

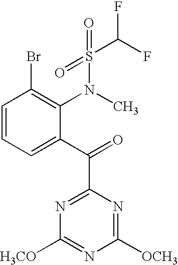

To a solution of 96 mg of N-{2-bromo-6-[(4,6-dimethoxy-1,3,5-triazin-2-yl)carbonyl]phenyl}-1,1-difluoromethanesulfonamide (0.212 mmol) in 3 ml of N,N-dimethylformamide, 44 mg of potassium carbonate (0.318 mmol) and 45 mg of iodomethane (0.218 mmol) were added. The resultant mixture was stirred for 6 hours at room temperature. To the reaction mixture, ethyl acetate and water were added. The resultant mixture was separated into an organic layer and an aqueous layer with a separating funnel. The organic layer was washed with water, dried with magnesium sulfate, and concentrated under a reduced pressure. The resultant residue was purified by column chromatography with a 3:2 mixed solvent of n-hexane and ethyl acetate to give 40 mg of desired product, N-{2-bromo-6-[(4,6-dimethoxy-1,3,5-triazin-2-yl)carbonyl]phenyl}-N-methyl-1,1-difluoromethanesulfonamide (yield: 40.4%).

¹H NMR (300 MHz, CDCl₃) δ 3.39 (3H, s), 4.11 (6H, s), 6.27 (1H, t), 7.38 (1H, t), 7.70 (1H, d), 7.88 (1H, d),

### Synthesis Example 8

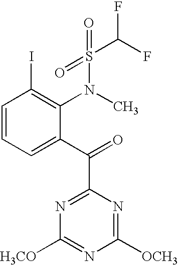

N-{2-iodo-6-[(4,6-dimethoxy-1,3,5-triazin-2-yl)carbonyl]phenyl}-N-methyl-1,1-difluoromethanesulfonamide was prepared by the method similar to of Synthesis Example 7.

¹H NMR (300 MHz, CDCl₃) 3.44 (3H, s), 4.10 (6H, s), 6.32 (1H, t), 7.20 (1H, t), 7.68 (1H, d), 8.14 (1H, d)

### Synthesis Example 9

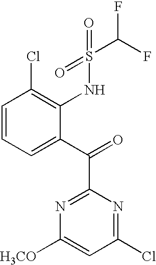

To a solution of 2.27 g of N-{2-chloro-6-[(4-chloro-6-methoxypyrimidin-2-yl)methyl]phenyl}-1,1-difluoromethanesulfonamide (5.70 mmol) in 25 ml of acetic acid, 2.28 g of chromic anhydride (22.80 mmol) was added at room temperature. The resultant mixture was stirred for one hour at room temperature. The reaction mixture was heated at 70° C. for five hours, ethyl acetate and water were added thereto. The resultant mixture was separated into an organic layer and an aqueous layer with a separating funnel. The organic layer was washed with water, dried with magnesium sulfate, and concentrated under a reduced pressure. The resultant residue was purified by column chromatography with a 1:1 mixed solvent of n-hexane and ethyl acetate to give 0.91 g of desired product, N-{2-chloro-6-[(4-chloro-6-methoxypyrimidin-2-yl)carbonyl]phenyl}-1,1-difluoromethanesulfonamide (yield: 38.7%).

¹H NMR (300 MHz, CDCl₃) δ4.04 (3H, s), 6.29 (1H, t), 6.91 (1H, s), 7.43 (1H, t), 7.66-7.73 (2H)

### Synthesis Example 10

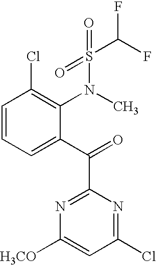

To a solution of 0.268 g of N-{2-chloro-6-[(4-chloro-6-methoxypyrimidin-2-yl)carbonyl]phenyl}-1,1-difluoromethanesulfonamide (0.65 mmol) in 4 ml of N,N-dimethylformamide, 0.135 g of potassium carbonate (0.975 mmol) and 0.12 g of methyl iodide (0.845 mmol) were added. The resultant mixture was stirred for four hours at room temperature. To the reaction mixture, ethyl acetate and water were added. The resultant blend was separated into an organic layer and an aqueous layer with a separating funnel. The organic layer was washed with water, dried with magnesium sulfate, and concentrated under a reduced pressure. The resultant residue was purified by column chromatography with a 2:1 mixed solvent of n-hexane and ethyl acetate to give 0.2 g of desired product, N-{2-chloro-6-[(4-chloro-6-methoxypyrimidin-2-yl)carbonyl]phenyl}-N-methyl-1,1-difluoromethanesulfonamide (yield: 72.2%).

¹H NMR (300 MHz, CDCl₃) δ 3.34 (3H, s), 4.06 (3H, s) 6.20 (1H, t), 6.90 (1H, s), 7.47 (1H, t), 7.64-7.73 (2H)

### Synthesis Example 11

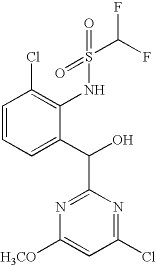

To a solution of 0.60 g of N-{2-chloro-6-[(4-chloro-6-methoxypyrimidin-2-yl)carbonyl]phenyl}-1,1-difluoromethanesulfonamide (1.456 mmol) in 7 ml of tetrahydrofuran and 3 ml of water, 0.10 g of sodium borohydride (2.62 mmol) was added under an ice-cooled condition. The resultant mixture was stirred for one hour. To the reaction mixture, ethyl acetate and water were added. The resultant blend was neutralized with 1N hydrochloric acid, and separated into an organic layer and an aqueous layer with a separating funnel. The organic layer was washed with water, dried with magnesium sulfate, and concentrated under a reduced pressure. The resultant residue was purified by column chromatography with a 2:1 mixed solvent of n-hexane and ethyl acetate to give 0.55 g of desired product, N-{2-chloro-6-[(4-chloro-6-methoxypyrimidin-2-yl)(hydroxyl)methyl]phenyl}-1,1-difluoromethanesulfonamide (yield: 91.7%).

¹H NMR (300 MHz, CDCl₃) δ 4.04 (3H, s), 5.14 (1H, br), 6.28 (1H, s) 6.73 (1H, t), 6.73 (1H, s), 7.45 (1H, t), 7.59 (1H), 7.62 (1H)

### Synthesis Example 12

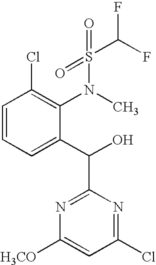

To a solution of 0.155 g of N-{2-chloro-6-[(4-chloro-6-methoxypyrimidin-2-yl)(hydroxyl)methyl]phenyl}-1,1-difluoromethanesulfonamide (0.37 mmol) in 4 ml of N,N-dimethylformamide, 0.047 g of sodium bicarbonate (0.56 mmol) and 0.080 g of methyl iodide (0.56 mmol) were added. The resultant mixture was stirred for four hours at room temperature. To the reaction mixture, ethyl acetate and water were added. The resultant blend was separated into an organic layer and an aqueous layer with a separating funnel. The organic layer was washed with water, dried with magnesium sulfate, and concentrated under a reduced pressure. The resultant residue was purified by column chromatography with a 5:3 mixed solvent of n-hexane and acetone to give 0.155 g of desired product, N-{2-chloro-6-[(4-chloro-6-methoxypyrimidin-2-yl)(hydroxyl)methyl]phenyl}-N-methyl-1,1-difluoromethanesulfonamide, as a rotamer mixture of a ratio of about 3:1 (yield: 96.7%).

¹H NMR (300 MHz, CDCl₃)

rotamer A

δ 3.56 (3H, s), 3.96 (3H, s), 4.40 (1H, d) 6.06 (1H, d) 6.53 (1H, t) 6.67 (1H, s) 7.2-7.35 (2H) 7.45 (1H)

rotamer B

δ 3.42 (3H, s), 3.94 (3H, s), 4.54 (1H, d) 6.02 (1H, d) 6.71 (1H, t) 6.98 (1H, s) 7.1-7.35 (2H) 7.45 (1H)

Compounds similarly prepared to in Synthesis Examples 1-12 are listed in the following Table 2, and physical and chemical properties thereof are listed in Table 3. In Table, Me, Et, and n-Pr represent methyl, ethyl, and n-propyl, respectively.

TABLE 2

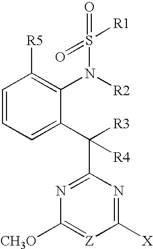

| Compound No. | R1 | R2 | R3 | R4 | R5 | Z | X |
|---|---|---|---|---|---|---|---|
| 1 | CH₂CF₃ | H | H | OH | Br | CH | OCH₃ |
| 2 | CH₂CF₃ | H | ═O | | Br | CH | OCH₃ |
| 3 | CH₂CF₃ | H | H | H | Br | CH | OCH₃ |
| 4 | CH₂CF₃ | H | H | SCH₃ | Br | CH | OCH₃ |
| 5 | CH₂CF₃ | H | H | H | I | CH | OCH₃ |
| 6 | CH₂CF₃ | H | ═O | | I | CH | OCH₃ |
| 7 | CH₂CF₃ | H | H | H | I | CH | OCH₃ |
| 8 | CH₂CF₃ | H | H | SCH₃ | I | CH | OCH₃ |
| 9 | CHF₂ | Me | H | OH | Br | N | OCH |
| 10 | CHF₂ | Me | ═O | | Br | N | OCH₃ |
| 11 | CHF₂ | Et | H | OH | Br | N | OCH₃ |
| 12 | CHF₂ | Et | ═O | | Br | N | OCH₃ |
| 13 | CHF₂ | n-Pr | H | OH | Br | N | OCH₃ |
| 14 | CHF₂ | n-Pr | ═O | | Br | N | OCH₃ |
| 15 | CHF₂ | 3-propenyl | H | OH | Br | N | OCH₃ |
| 16 | CHF₂ | 3-propenyl | ═O | | Br | N | OCH₃ |
| 17 | CHF₂ | 3-propynyl | H | OH | Br | N | OCH₃ |
| 18 | CHF₂ | 3-propynyl | ═O | | Br | N | OCH₃ |
| 19 | CHF₂ | Me | H | OH | I | N | OCH₃ |
| 20 | CHF₂ | Me | ═O | | I | N | OCH₃ |
| 21 | CHF₂ | Et | H | OH | I | N | OCH₃ |
| 22 | CHF₂ | Et | ═O | | I | N | OCH₃ |
| 23 | CHF₂ | n-Pr | H | OH | I | N | OCH₃ |
| 24 | CHF₂ | n-Pr | ═O | | I | N | OCH₃ |
| 25 | CHF₂ | 3-propenyl | H | OH | I | N | OCH₃ |
| 26 | CHF₂ | 3-propenyl | ═O | | I | N | OCH₃ |
| 27 | CHF₂ | 3-propynyl | H | OH | I | N | OCH₃ |
| 28 | CHF₂ | 3-propynyl | ═O | | I | N | OCH₃ |
| 29 | CHF₂ | H | H | OH | F | CH | Cl |
| 30 | CHF₂ | H | ═O | | F | CH | Cl |
| 31 | CHF₂ | H | H | H | F | CH | Cl |
| 32 | CHF₂ | Me | H | OH | F | CH | Cl |
| 33 | CHF₂ | Me | ═O | | F | CH | Cl |
| 34 | CHF₂ | Me | H | H | F | CH | Cl |
| 35 | CHF₂ | Et | H | OH | F | CH | Cl |
| 36 | CHF₂ | Et | ═O | | F | CH | Cl |
| 37 | CHF₂ | Et | H | H | F | CH | Cl |
| 38 | CHF₂ | n-Pr | H | OH | F | CH | Cl |
| 39 | CHF₂ | n-Pr | ═O | | F | CH | Cl |
| 40 | CHF₂ | n-Pr | H | H | F | CH | Cl |
| 41 | CHF₂ | 3-propenyl | H | OH | F | CH | Cl |
| 42 | CHF₂ | 3-propenyl | ═O | | F | CH | Cl |
| 43 | CHF₂ | 3-propenyl | H | H | F | CH | Cl |
| 44 | CHF₂ | 3-propynyl | H | OH | F | CH | Cl |
| 45 | CHF₂ | 3-propynyl | ═O | | F | CH | Cl |
| 46 | CHF₂ | 3-propynyl | H | H | F | CH | Cl |
| 47 | CHF₂ | H | H | OH | Cl | CH | Cl |
| 48 | CHF₂ | H | ═O | | Cl | CH | Cl |
| 49 | CHF₂ | H | H | H | Cl | CH | Cl |
| 50 | CHF₂ | Me | H | OH | Cl | CH | Cl |
| 51 | CHF₂ | Me | ═O | | Cl | CH | Cl |
| 52 | CHF₂ | Me | H | H | Cl | CH | Cl |
| 53 | CHF₂ | Et | H | OH | Cl | CH | Cl |
| 54 | CHF₂ | Et | ═O | | Cl | CH | Cl |
| 55 | CHF₂ | Et | H | H | Cl | CH | Cl |
| 56 | CHF₂ | n-Pr | H | OH | Cl | CH | Cl |
| 57 | CHF₂ | n-Pr | ═O | | Cl | CH | Cl |
| 58 | CHF₂ | n-Pr | H | H | Cl | CH | Cl |
| 59 | CHF₂ | 3-propenyl | H | OH | Cl | CH | Cl |
| 60 | CHF₂ | 3-propenyl | ═O | | Cl | CH | Cl |
| 61 | CHF₂ | 3-propenyl | H | H | Cl | CH | Cl |
| 62 | CHF₂ | 3-propynyl | H | OH | Cl | CH | Cl |
| 63 | CHF₂ | 3-propynyl | ═O | | Cl | CH | Cl |
| 64 | CHF₂ | 3-propynyl | H | H | Cl | CH | Cl |
| 65 | CHF₂ | H | H | OH | Br | CH | Cl |
| 66 | CHF₂ | H | ═O | | Br | CH | Cl |
| 67 | CHF₂ | H | H | H | Br | CH | Cl |
| 68 | CHF₂ | Me | H | OH | Br | CH | Cl |
| 69 | CHF₂ | Me | ═O | | Br | CH | Cl |
| 70 | CHF₂ | Me | H | H | Br | CH | Cl |
| 71 | CHF₂ | Et | H | OH | Br | CH | Cl |
| 72 | CHF₂ | Et | ═O | | Br | CH | Cl |
| 73 | CHF₂ | Et | H | H | Br | CH | Cl |
| 74 | CHF₂ | n-Pr | H | OH | Br | CH | Cl |
| 75 | CHF₂ | n-Pr | ═O | | Br | CH | Cl |
| 76 | CHF₂ | n-Pr | H | H | Br | CH | Cl |
| 77 | CHF₂ | 3-propenyl | H | OH | Br | CH | Cl |
| 78 | CHF₂ | 3-propenyl | ═O | | Br | CH | Cl |
| 79 | CHF₂ | 3-propenyl | H | H | Br | CH | Cl |
| 80 | CHF₂ | 3-propynyl | H | OH | Br | CH | Cl |
| 81 | CHF₂ | 3-propynyl | ═O | | Br | CH | Cl |
| 82 | CHF₂ | 3-propynyl | H | H | Br | CH | Cl |
| 83 | CHF₂ | H | H | OH | I | CH | Cl |
| 84 | CHF₂ | H | ═O | | I | CH | Cl |
| 85 | CHF₂ | H | H | H | I | CH | Cl |
| 86 | CHF₂ | Me | H | OH | I | CH | Cl |
| 87 | CHF₂ | Me | ═O | | I | CH | Cl |
| 88 | CHF₂ | Me | H | H | I | CH | Cl |
| 89 | CHF₂ | Et | H | OH | I | CH | Cl |
| 90 | CHF₂ | Et | ═O | | I | CH | Cl |
| 91 | CHF₂ | Et | H | H | I | CH | Cl |
| 92 | CHF₂ | n-Pr | H | OH | I | CH | Cl |
| 93 | CHF₂ | n-Pr | ═O | | I | CH | Cl |
| 94 | CHF₂ | n-Pr | H | H | I | CH | Cl |
| 95 | CHF₂ | 3-propenyl | H | OH | I | CH | Cl |
| 96 | CHF₂ | 3-propenyl | ═O | | I | CH | Cl |
| 97 | CHF₂ | 3-propenyl | H | H | I | CH | Cl |
| 98 | CHF₂ | 3-propynyl | H | OH | I | CH | Cl |
| 99 | CHF₂ | 3-propynyl | ═O | | I | CH | Cl |
| 100 | CHF₂ | 3-propynyl | H | H | I | CH | Cl |
| 101 | CHF₂ | H | H | OH | Me | CH | Cl |
| 102 | CHF₂ | H | ═O | | Me | CH | Cl |
| 103 | CHF₂ | H | H | H | Me | CH | Cl |
| 104 | CHF₂ | Me | H | OH | Me | CH | Cl |
| 105 | CHF₂ | Me | ═O | | Me | CH | Cl |
| 106 | CHF₂ | Me | H | H | Me | CH | Cl |
| 107 | CHF₂ | Et | H | OH | Me | CH | Cl |
| 108 | CHF₂ | Et | ═O | | Me | CH | Cl |
| 109 | CHF₂ | Et | H | H | Me | CH | Cl |
| 110 | CHF₂ | n-Pr | H | OH | Me | CH | Cl |
| 111 | CHF₂ | n-Pr | ═O | | Me | CH | Cl |
| 112 | CHF₂ | n-Pr | H | H | Me | CH | Cl |
| 113 | CHF₂ | 2-propenyl | H | OH | Me | CH | Cl |
| 114 | CHF₂ | 2-propenyl | ═O | | Me | CH | Cl |
| 115 | CHF₂ | 2-propenyl | H | H | Me | CH | Cl |
| 116 | CHF₂ | 2-propynyl | H | OH | Me | CH | Cl |
| 117 | CHF₂ | 2-propynyl | ═O | | Me | CH | Cl |
| 118 | CHF₂ | 2-propynyl | H | H | Me | CH | Cl |

TABLE 3

| Compound | Physical property (1H-NMR (300 MHz, CDCl3) δ) or |
|---|---|
| No. | melting point (° C.) |
| 1 | 3.97(6H, s), 4.10-4.24(1H, m), 4.65-4.79(1H, m), |
| | 5.00(1H, s), 5.98(1H, m), 6.30(1H, d), 7.20(1H, t), |
| | 7.58(1H, dd), 7.68(1H, dd), 10.01(1H, br) |
| 2 | 3.94(6H, s), 4.06(2H, q), 6.18(1H, s), 7.32(1H, t), |
| | 7.60(1H, br), 7.69(1H, dd), 7.88(1H, dd) |
| 4 | 2.04(3H, s), 3.94(6H, s), 4.08-4.29(1H, m), |
| | 4.61-4.75(1H, m), 5.81(1H, s), 5.90(1H, s), 7.24(1H, t), |
| | 7.56(1H, dd), 8.06(1H, dd), 9.16(1H, br) |
| 5 | 3.97(6H, s), 4.20-4.30(1H, m), 4.79-4.93(1H, m), |
| | 5.00(1H, s), 5.97(1H, m), 6.36(1H, d), 7.07(1H, t), |
| | 7.69(1H, dd), 7.83(1H, dd), 10.00(1H, br) |
| 6 | 3.93(6H, s), 4.06(2H, q), 6.18 (1H, s), 7.17(1H, t), |
| | 7.59(1H, br), 7.68(1H, dd), 8.10(1H, dd) |
| 8 | 2.04(3H, s), 3.94(6H, s), 4.23-4.31(1H, m), |
| | 4.80-4.87(1H, m), 5.89(1H, s), 5.90(1H, s), 7.08(1H, t), |
| | 7.82(1H, dd), 8.09(1H, dd), 9.14(1H, br) |
| 10 | 3.39(3H, s), 4.11(6H, s), 6.27(1H, t), 7.39(1H, t), |
| | 7.69(1H, d), 7.88(1H, d) |
| 20 | 3.44(3H, s), 4.10(6H, s), 6.32(1H, t), 7.20(1H, t), |
| | 7.68(1H, d), 8.14(1H, d) |
| 31 | 4.03(3H, s), 4.32(2H, s), 6.57(1H, t), 6.75(1H, s), |
| | 7.05-7.30(3H), 10.23(1H, s) |
| 47 | 4.04(3H, s), 5.14(1H, br), 6.28(1H, s) 6.73(1H, t), |
| | 6.73(1H, s), 7.45(1H, t), 7.59 (1H), 7.62(1H) |
| 48 | 4.04(3H, s), 6.29(1H, t), 6.91(1H, s), 7.43(1H, t), |
| | 7.66-7.73(2H) |
| 49 | 4.02(3H, s), 4.38(2H, s), 6.67(1H, s), 6.71(1H, t), |
| | 7.18-7.50(3H), 10.07(1H, s) |
| 50 | rotamer A |
| | 3.56(3H, s), 3.96(3H, s), 4.40(1H, d) 6.06(1H, d) |
| | 6.53(1H, t) 6.67(1H, s) 7.2-7.35(2H) 7.45(1H) |
| | rotamer B |
| | 3.42(3H, s), 3.94(3H, s), 4.54(1H, d) 6.02(1H, d) |
| | 6.71(1H, t) 6.98(1H, s) 7.1-7.35(2H) 7.45(1H) |
| 51 | 3.34(3H, s), 4.06(3H, s) 6.20(1H, t), 6.90(1H, s), |
| | 7.47(1H, t), 7.64-7.73(2H) |

### Synthesis Example 13

### Synthesis of Intermediate

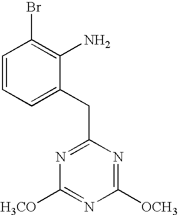

A solution of 2.30 g 2-bromoaniline (13.37 mmol) in 30 ml of methylene chloride was cooled to a temperature of −65° C. or less. To this, a solution of 1.60 g of tert-butyl hypochlorite (14.07 mmol) in methylene chloride (5 ml) was added dropwise. The resultant was stirred for 10 minutes at a temperature of −65° C. or lower. To the resultant mixture, 2.69 g of 2-methylthiomethyl-4,6-dimethoxytriazine (13.37 mmol) in methylene chloride (10 ml) was added dropwise. The resultant blend was stirred for one hour at a temperature of −65° C. or lower. To the reaction mixture, a solution of 1.76 g of triethylamine (17.38 mmol) in methylene chloride (10 ml) was added dropwise at a temperature of −65° C. or lower. The resultant mixture was stirred for 30 minutes, and stirred until the temperature reached room temperature. To the reaction mixture, water was added. An organic layer was separated with a separation funnel, and an aqueous layer was extracted with methylene chloride. The organic layer was washed with water again, dried with magnesium sulfate, and concentrated under a reduced pressure to give 5.0 g of oily crude product of 2-bromo-6-[(4,6-dimethoxy-1,3,5-triazin-2-yl)(methylthio)methyl]aniline.

5.0 g of crude product of 2-bromo-6-[(4,6-dimethoxy-1,3,5-triazin-2-yl)(methylthio)methyl]aniline (13.47 mmol) and 3.20 g of nickel(II) chloride hexahydrate (13.47 mmol) were dissolved in methanol (50 ml). To the solution, which was being stirred, 1.02 g of sodium borohydride (26.94 mmol) was gradually added under an ice-cooled condition. The reaction mixture was stirred for one hour at room temperature, and ethyl acetate and ammonia water were added thereto. Insoluble matters were filtered out. The filtrate was separated into an organic layer and an aqueous layer with a separation funnel. The organic layer was washed with water, dried with magnesium sulfate, and concentrated under a reduced pressure to give an oily crude product.

The resultant crude product was purified by column chromatography with a 3:1 mixed solvent of n-hexane and ethyl acetate to give 3.50 g of desired product, 2-bromo-6-[(4,6-dimethoxy-1,3,5-triazin-2-yl)methyl]aniline (yield: 79.9%).

¹H NMR (300 MHz, CDCl₃) δ 3.98 (2H, s), 4.03 (6H, s), 5.07 (2H, s), 6.58 (1H, t), 7.20 (1H, d), 7.33 (1H, d)

### Synthesis Example 14

### Synthesis of Intermediate

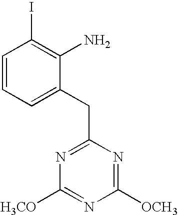

2-Iodo-6-[(4,6-dimethoxy-1,3,5-triazin-2-yl)methyl]aniline was prepared by the similar method to Synthesis Example 12.

¹H NMR (300 MHz, CDCl₃) δ 3.99 (2H, s), 4.02 (6H, s), 5.08 (2H, s) 6.45 (1H, t), 7.21 (1H, d), 7.55 (1H, d)

### Synthesis Example 15

### Synthesis of Intermediate

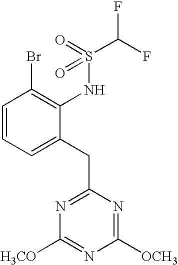

A solution of 3.40 g of 2-bromo-6-[(4,6-dimethoxy-1,3,5-triazin-2-yl)methyl]aniline (10.46 mmol) and 1.65 g of pyridine (20.91 mmol) in methylene chloride (6 ml) was cooled to −30° C. To this, a solution of 3.15 g of difluoromethanesulfonyl chloride (20.91 mmol) in methylene chloride (6 ml) was added dropwise. Then the reaction mixture was returned to room temperature, and stirred overnight. To the reaction mixture, water was added. The resultant mixture was separated into an organic layer and an aqueous layer with a separation funnel. The organic layer was washed with water, dried with magnesium sulfate, and concentrated under a reduced pressure. The resultant residue was purified by column chromatography with a 3:2 mixed solvent of n-hexane and ethyl acetate to give 1.10 g of desired product, N-{2-bromo-6-[(4,6-dimethoxy-1,3,5-triazin-2-yl)methyl]phenyl}-1,1-difluoromethanesulfonamide (yield: 24%).

¹H NMR (300 MHz, CDCl₃) δ 3.88 (1H, d), 4.02 (6H, s), 4.60 (1H, d), 6.52 (1H, t), 7.04 (1H, t), 7.32 (1H, d), 7.54 (1H, d), 9.36 (1H, s)

### Synthesis Example 16

### Synthesis of Intermediate

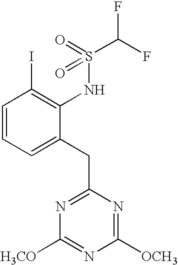

N-{2-[(4,6-Dimethoxy-1,3,5-triazin-2-yl)methyl]-6-iodophenyl}-1,1-difluoromethanesulfonamide was prepared by the similar method to Synthesis Example 14.

¹H NMR (300 MHz, CDCl₃) δ 3.85 (1H, d), 4.02 (6H, s), 4.71 (1H, d), 6.62 (1H, t), 6.89 (1H, t), 7.35 (1H, d), 7.80 (1H, d), 9.53 (1H, s)

### Synthesis Example 17

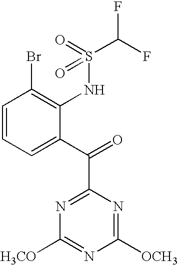

To a solution of 1.10 g of N-{2-bromo-6-[(4,6-dimethoxy-1,3,5-triazin-2-yl)methyl]phenyl}-1,1-difluoromethanesulfonamide (2.50 mmol) in acetic acid (10 ml), 1.00 g of chromic anhydride (10.02 mmol) was added. The resultant mixture was stirred for 16 hours. To the reaction mixture, ethyl acetate and water were added. The resultant blend was separated into an organic layer and an aqueous layer with a separation funnel. The aqueous layer was extracted with ethyl acetate again. The organic layer was washed with water, dried with magnesium sulfate, and concentrated under a reduced pressure. The residue was dissolved in ethyl acetate, an aqueous potassium carbonate solution was added thereto, and the resultant admixture was separated into an organic layer and an aqueous layer with a separation funnel. The aqueous layer was acidified with 1N hydrochloric acid, and extracted with ethyl acetate. The separated ethyl acetate layer was dried with magnesium sulfate, and concentrated under a reduced pressure to give 0.17 g of desired product, N-{2-bromo-6-[(4,6-dimethoxy-1,3,5-triazin-2-yl)carbonyl]phenyl}-1,1-difluoromethanesulfonamide (yield: 15.0%).

¹H NMR (300 MHz, CDCl₃) δ 4.08 (6H, s), 6.27 (1H, t), 7.34 (1H, t), 7.74 (1H, d), 7.87 (1H, d)

### Synthesis Example 18

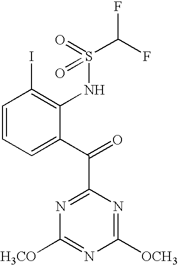

N-{2-Iodo-6-[(4,6-dimethoxy-1,3,5-triazin-2-yl)carbonyl]phenyl}-1,1-difluoromethanesulfonamide was prepared by the similar method to Synthesis Example 16.

¹H NMR (300 MHz, CDCl₃) δ 4.11 (6H, s), 6.31 (1H, t), 7.19 (1H, t), 7.77 (1H, d), 8.15 (1H, d)

### Synthesis Example 19

### Synthesis of Intermediate

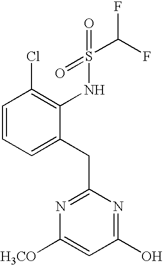

To a solution of 3.00 g of N-{2-chloro-6-[(4,6-dimethoxypyrimidin-2-yl)methyl]phenyl}-1,1-difluoromethanesulfonamide (7.62 mmol) in 30 ml of acetone, an aqueous 47% hydrogen bromide solution was added under an ice-cooled condition. The reaction mixture was refluxed for two hours, and cooled to room temperature. The reaction mixture was concentrated under a reduced pressure, and water was added thereto to precipitate a solid. The solid was collected by filtration, washed with water, and dried to give 2.37 g of desired product, N-{2-chloro-6-[(4-hydroxy-6-methoxypyrimidin-2-yl)methyl]phenyl}-1,1-difluoromethanesulfonamide (yield: 82%).

¹H NMR (300 MHz, acetone-d₆) δ 3.77 (3H, s), 4.29 (2H, s), 5.45 (1H, s), 6.97 (1H, t), 7.25-7.60 (3H)

### Synthesis Example 20

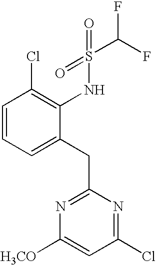

2.35 g of N-{2-chloro-6-[(4-hydroxy-6-methoxypyrimidin-2-yl)methyl]phenyl}-1,1-difluoromethanesulfonamide (6.19 mmol) and 0.75 g of N,N-dimethylaniline (6.19 mmol) were added to 15 ml of phosphorus oxychloride. The reaction mixture was heated at 120° C. for two hours. The reaction mixture was poured into water which was being stirred. After phosphorus oxychloride was decomposed, ethyl acetate was added to the reaction mixture, and the resultant was separated into an organic layer and an aqueous layer with a separation funnel. The organic layer was washed with water, dried with magnesium sulfate, and concentrated under a reduced pressure. The resultant residue was purified by column chromatography with a 3:1 mixed solvent of n-hexane and ethyl acetate to give 2.42 g of desired product, N-{2-chloro-6-[(4-chloro-6-methoxypyrimidin-2-yl)methyl]phenyl}-1,1-difluoromethanesulfonamide (yield: 98.2%).

¹H NMR (300 MHz, CDCl₃) δ4.02 (3H, s), 4.38 (2H, s), 6.67 (1H, s), 6.71 (1H, t), 7.18-7.50 (3H), 10.07 (1H, s)

### Synthesis Example 21

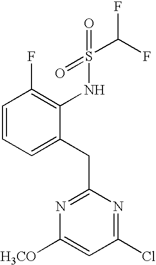

N-{2-Fluoro-6-[(4-chloro-6-methoxypyrimidin-2-yl)methyl]phenyl}-1,1-difluoromethanesulfonamide was prepared by the similar method to Synthesis Example 19.

¹H NMR (300 MHz, CDCl₃) δ4.03 (3H, s), 4.32 (2H, s), 6.57 (1H, t), 6.75 (1H, s), 7.05-7.30 (3H), 10.23 (1H, s)

### Test Example

Test of herbicidal effect to weeds and damage on paddy rice in a paddy field: In a greenhouse, each three paddy rice seedlings (Oryza sativa L. cultivar: Nihonbare) were transplanted in each pot filled with paddy field soil. Each twenty forced sprouts of paddy rice (Oryza sativa L. cultivar: Nihonbare) were also inoculated in each pot. Then, any of seeds or tubers of Cyperus difformis L., Echinochloa crus-galli, Scirpus juncoides, SU-resistant Scirpus juncoides, annual broad leaf weeds (including Lindernia procumbens Philcox, Rotala indica Koehne, and Elatine triandra Schk), SU-resistant annual broad leaf weeds, Sagittaria pygmaea Miq, Cyperus serotinus Rottboel was inoculated in the pot and covered with water in about 2 to 3 cm depth. Five days after transplanting paddy rice, prescribed diluted solutions of formulations of the respective active compounds were applied onto the water surface. Each of the formulations was prepared as an emulsion by mixing 1 part by weight of active compound with 5 parts by weight of carrier and 1 part by weight of emulsifier (benzyloxy polyglycol ether). After the treatment, water depth of 3 cm was kept. A herbicidal effect and a herbicidal damage on paddy rice were investigated 3 weeks after the treatment. The herbicidal effect and the herbicidal damage on paddy rice were rated as 100% in the case of complete withering and 0% in the case of no herbicidal effect or no herbicidal damage.

TABLE 4

| | Rate | | directley | | | | SU-resistant | | SU-resistant | |
|---|---|---|---|---|---|---|---|---|---|---|
| Compound | (g | transplanted | seeded | Cyperus | Echinochloa | Sagittaria | Scirpus | Scirpus | annual broad | annual broad |
| No. | ai/ha) | rice | rice | serotinus | crus-galli | pygmaea | juncoides | juncoides | leaf weeds | leaf weeds |
| 10 | 125 | 10 | 30 | 100 | 100 | 100 | 90 | 100 | 100 | 90 |
| 4 | 125 | 0 | 10 | 100 | 100 | 100 | 90 | 100 | 100 | 100 |
| 2 | 125 | 10 | 20 | 100 | 100 | 100 | 100 | 100 | 100 | 100 |
| 1 | 125 | 0 | 10 | 100 | 100 | 100 | 100 | 100 | 100 | 100 |
| 8 | 125 | 0 | 10 | 100 | 100 | 100 | 90 | 100 | 100 | 100 |
| 5 | 125 | 20 | 30 | 100 | 100 | 100 | 90 | 100 | 100 | 100 |
| 51 | 125 | 10 | 30 | 100 | 100 | 100 | 90 | 100 | 80 | 90 |

1. A herbicide comprising as an active ingredient a sulfonanilide compound represented by the formula: 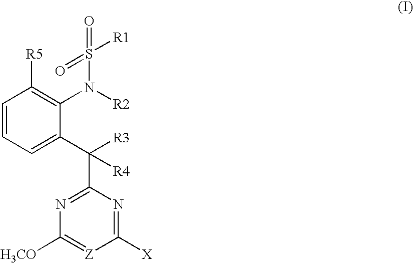
wherein,
R1 represents CHF₂ or CH₂CF₃,
R2 represents hydrogen, C1-3 alkyl, 3-propenyl, or 3-propynyl,
R3 represents hydrogen,
R4 represents hydrogen, hydroxy, or methylthio,
R3 and R4, together with a carbon atom to which they are bonded, may form C═O,
R5 represents halogen or methyl,
X represents methoxy or chlorine, and
Z represents CH or N,
with the proviso that,
(i) when R1 represents CH₂CF₃, R2 represents hydrogen, R5 represents bromine or iodine, X represents methoxy, and Z represents CH,
(ii) when R1 represents CHF₂ and X represents methoxy, R5 represents bromine or iodine, Z represents N, and R2 represents C1-3 alkyl, 3-propenyl, or 3-propynyl,
(iii) when R1 represents CHF₂ and X represents chlorine, Z represents CH.

2. The herbicide according to claim 1, wherein
R1 represents CH₂CF₃,
R2 represents hydrogen,
R3 represents hydrogen,
R4 represents hydrogen, hydroxy, or methylthio, R3 and R4, together with a carbon atom to which they are bonded, may form C═O,
R5 represents bromine or iodine,
X represents methoxy, and
Z represents CH.

3. The herbicide according to claim 1, wherein
R1 represents CHF₂,
R2 represents C1-3 alkyl, 3-propenyl, or 3-propynyl,
R3 represents hydrogen,
R4 represents hydrogen or hydroxy,
R3 and R4, together with a carbon atom to which they are bonded, may form C═O,
R5 represents bromine or iodine,
X represents methoxy, and
Z represents N.

4. The herbicide according to claim 1, wherein
R1 represents CHF₂,
R2 represents hydrogen, C1-3 alkyl, 3-propenyl, or 3-propynyl,
R3 represents hydrogen,
R4 represents hydrogen or hydroxy,
R3 and R4, together with a carbon atom to which they are bonded, may form C═O,
R5 represents fluorine, chlorine, bromine, iodine or methyl,
X represents chlorine, and
Z represents CH.

5. A sulfonanilide compound represented by the formula: 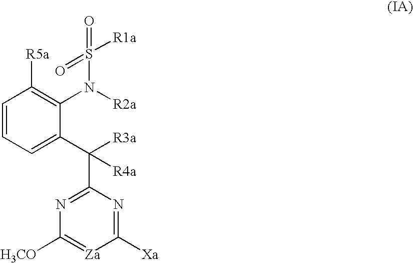
wherein,
R1a represents CH₂CF₃,
R2a represents hydrogen,
R3a represents hydrogen,
R4a represents hydrogen, hydroxy, or methylthio,
R3a and R4a, together with a carbon atom to which they are bonded, may form C═O,
R5a represents bromine or iodine,
Xa represents methoxy, and
Za represents CH.

6. A sulfonanilide compound represented by the formula: 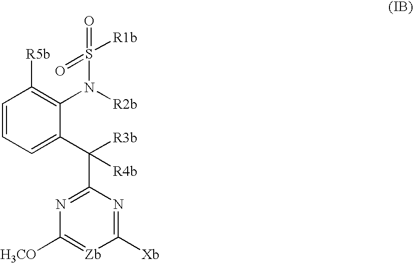
wherein,
R1b represents CHF₂,
R2b represents methyl, ethyl, propyl, 3-propenyl, or 3-propynyl,
R3b represents hydrogen,
R4b represents hydrogen or hydroxy,
R3b and R4b, together with a carbon atom to which they are bonded, may form C═O,
R5b represents bromine or iodine,
Xb represents methoxy, and
Zb represents N.

7. A sulfonanilide compound represented by the formula: 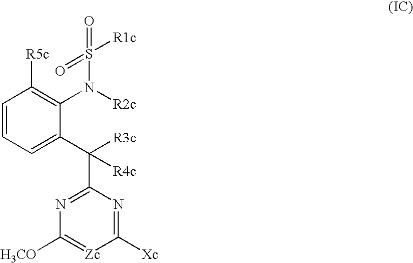
wherein, R1c represents CHF₂,
R2c represents hydrogen, methyl, ethyl, propyl, 3-propenyl, or 3-propynyl,
R3c represents hydrogen,
R4c represents hydrogen or hydroxy,
R3c and R4c, together with a carbon atom to which they are bonded, may form C═O,
R5c represents fluorine, chlorine, bromine, iodine, or methyl,
Xc represents chlorine, and
Zc represents CH,
with the proviso that,
when R2c represents hydrogen, R5c represents bromine, iodine, or methyl.
